(19) United States
(12) Patent Application Publication
(10) Pub. No.: US 2019/0336636 A1
(43) Pub. Date: Nov. 7, 2019

(54) DISSOLVABLE EFFERVESCENT TOILET DEODORIZER AND METHOD OF USE

(71) Applicants: Taralee Gurr, St. George, UT (US); Robert A. Gurr, St. George, UT (US)

(72) Inventors: Taralee Gurr, St. George, UT (US); Robert A. Gurr, St. George, UT (US)

(21) Appl. No.: 16/404,596

(22) Filed: May 6, 2019

Related U.S. Application Data
(60) Provisional application No. 62/667,036, filed on May 4, 2018.
(60) Provisional application No. 62/819,516, filed on Mar. 15, 2019.

(51) Int. Cl. A61L 9/05; A61L 9/04

(52) U.S. Cl. CPC A61L 9/05; A61L 2209/21; A61L 9/042; A61L 9/046

(57) ABSTRACT

A dissolvable effervescent toilet deodorizer includes a combination of citric acid, sodium bicarbonate, and essential oils. In one method of use, a user would drop the dissolvable effervescent toilet deodorizer into the toilet bowl just prior to using the toilet. Once dropped into the toilet bowl, the dissolvable effervescent deodorizer begins to dissolve and effervesce. As a result of the effervescence, the dissolution occurs more rapidly and also disperses the deodorizer/scent into the toilet water and air to more effectively neutralize and/or prevent malodors.

### CROSS-REFERENCE TO RELATED APPLICATIONS

This application claims the benefit of U.S. Provisional Application Ser. No. 62/667,036, filed on May 4, 2018, and further claims the benefit of U.S. Provisional Application Ser. No. 62/819,516, filed on Mar. 15, 2019, both of which are incorporated herein by reference.

### TECHNICAL FIELD

The disclosure relates to toilet deodorizers. More particularly, the present disclosure relates to a dissolvable effervescent toilet deodorizer (e.g., tablet or strip) for use in a toilet while a user uses the toilet.

### BACKGROUND

Conventional deodorizers come in many varieties, with the most well-known being a liquid spray. The liquid spray may be dispersed into the air, or directly onto an odorous ornament or substance. More recently, sprays have been used to spray directly into a toilet before use. These sprays have become increasingly popular and are effective at reducing or eliminating malodors that emanate from the toilet during human elimination. However, the spray bottles are inconvenient to transport and there is a risk of the bottle leaking its contents. Because of this, many users do not carry the spray with them. As such, there remains a need for a deodorizer that is not in liquid form and that can be easily transported.

Further, as the contents of spray bottles decrease, spraying becomes difficult due to the angle and position of the straw and liquid in the bottle. This is especially a problem when the bottle is held horizontally for spraying into a toilet, as the end of the straw will not be in the remaining liquid. As such, there is a need for a deodorizer that is not dependent upon the angle of the container or fill level of the container.

Yet another problem is the ability to use spray deodorizers after a person has commenced elimination. Current spray bottles are ineffective in this scenario as a user is sitting on the toilet. Accordingly, there remains a need for a deodorizer that can be added to the toilet at any time during use.

The present disclosure seeks to solve these and other problems.

### SUMMARY OF EXAMPLE EMBODIMENTS

In one embodiment, a dissolvable effervescent toilet deodorizer comprises citric acid, sodium bicarbonate, microcrystalline cellulose, silica, and one or more essential oils. The effervescent toilet deodorizer may in the form of tablet for dropping in the toilet bowl.

In one embodiment, a dissolvable effervescent toilet deodorizer comprises citric acid, sodium bicarbonate, and a deodorizer.

In one embodiment, a dissolvable effervescent toilet deodorizer comprises baking soda, citric acid, and cornstarch or arrowroot.

In one embodiment, a dissolvable effervescent toilet deodorizer comprises a water-soluble strip of material comprising essential oils.

In one embodiment, a dissolvable effervescent toilet deodorizer comprises a water-soluble strip of material comprising citric acid, sodium bicarbonate, and essential oils.

In one embodiment, a dissolvable effervescent toilet deodorizer comprises a water-soluble strip of material comprising citric acid, sodium bicarbonate, and a deodorizer.

In one embodiment, a dissolvable effervescent toilet deodorizer comprises a water-soluble pouch, the pouch comprising citric acid, sodium bicarbonate, and a deodorizer.

In one embodiment, a dissolvable effervescent toilet deodorizer comprises a water-soluble strip of material comprising citric acid, sodium bicarbonate, and a deodorizer affixed to the exterior of the water-soluble strip of material.

In one embodiment a dissolvable effervescent toilet deodorizer comprises a water-soluble strip of material comprising a deodorizer. In one embodiment, the deodorizer comprises an essential oil.

In one method of use, a user would drop a dissolvable effervescent toilet deodorizer into the toilet bowl just prior to use. Once dropped into the toilet bowl, the dissolvable effervescent deodorizer begins to dissolve and effervesce. The dissolution occurs more rapidly as a result of the effervescence and also disperses into the water and air to more effectively neutralize and/or prevent malodors.

### DETAILED DESCRIPTION OF EXAMPLE EMBODIMENTS

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” does not necessarily refer to the same embodiment, although it may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various different sequences and arrangements while still falling within the scope of the present invention.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

As discussed above, there is a need for a toilet deodorizer that 1) is not in liquid form; 2) allows for easy storage and use; 3) that can be added to the toilet at any time during use; and 4) that effectively eliminates malodors. The dissolvable effervescent toilet deodorizer disclosed herein solves these, and other, problems.

In one embodiment, a dissolvable effervescent toilet deodorizer comprises a composition comprising citric acid, sodium bicarbonate, microcrystalline cellulose (MCC), silica, and one or more essential oils (e.g., lemongrass). Accordingly, the effervescent toilet deodorizer may in the form of tablet for dropping in the toilet bowl. In one non-limiting example, the dissolvable effervescent toilet deodorizer comprises the contents and amounts as listed in Table 1 below:

TABLE 1

| Description | % | mg/tab | Qty (g) |
|---|---|---|---|
| Sodium Bicarbonate | 18.33% | 595.8000 | 3.666462 |
| Citric Acid | 13.98% | 454.2000 | 2.795077 |
| MCC | 21.54% | 700.0000 | 4.307692 |
| Silica | 30.77% | 1000.0000 | 6.153846 |
| Essential Oil | 15.38% | 500.0000 | 3.076923 |

It will be appreciated that Table 1 illustrates example amounts only and the dissolvable effervescent toilet deodorizer is not limited to those amounts and percentages. In one non-limiting example, the amount of essential oil may be increased so as to more effectively counter-act the malodor. In such as scenario, it may be necessary to increase the silica and other dry compounds to offset the increased oil concentration. Those of skill in the art of tablet manufacturing will readily appreciate and understand how to adjust the balances. One in the art would further recognize that lubricants may also be needed, such as magnesium stearate, to more effectively release the tablets from the tablet press.

In one embodiment, a dissolvable effervescent toilet deodorizer comprises citric acid, sodium bicarbonate, and a deodorizer. In one embodiment, the deodorizer may comprise ozone. In one embodiment, the deodorizer comprises a biocide.

In one embodiment, a dissolvable effervescent toilet deodorizer comprises baking soda, citric acid, and cornstarch or arrowroot. In one embodiment, a dissolvable effervescent toilet deodorizer comprises dehydrated vinegar.

As will be appreciated from the foregoing, a dissolvable effervescent toilet deodorizer may take the formfactor of a tablet. This tablet is easily transportable, can be stored for long periods of time, can be easily dropped into a toilet just prior to use, and may be dropped into the toilet during use as well. This overcomes the issues found in the prior art. However, the dissolvable effervescent toilet deodorizer is not limited to tablets. For example, in one embodiment, the dissolvable effervescent toilet deodorizer may be in the formfactor of a dissolvable strip. Much like the benefits of the tablet over liquid, the strip allows a user to carry the deodorizer in a discrete manner, without fear of leakage, and that allows for increased dispersing and scent. Further, due to the thin formfactor, a user can carry a significant number of strips without losing precious space, such as in a user's pocket or purse. In one embodiment, the dissolvable strips may be in similar size to Listerine® breath strips, allowing a user to carry several strips conveniently. Accordingly, a dissolvable strip for use in the toilet has many benefits over the prior art.

In one embodiment, a dissolvable effervescent toilet deodorizer comprises a water-soluble strip of material comprising essential oils.

In one embodiment, a dissolvable effervescent toilet deodorizer comprises a water-soluble strip of material comprising citric acid, sodium bicarbonate, and essential oils.

In one embodiment, a dissolvable effervescent toilet deodorizer comprises a water-soluble strip of material comprising citric acid, sodium bicarbonate, and a deodorizer.

In one embodiment, a dissolvable effervescent toilet deodorizer comprises a water-soluble pouch, the pouch comprising citric acid, sodium bicarbonate, and a deodorizer.

In one embodiment, a dissolvable effervescent toilet deodorizer comprises a water-soluble strip of material comprising citric acid, sodium bicarbonate, and a deodorizer, with at least the citric acid and sodium bicarbonate affixed to, or embedded in, the exterior of the water-soluble strip of material. Having the citric acid and sodium bicarbonate on the exterior of the strip of material allows the particles to come into contact with the water faster, thereby effervescing. As a result of the effervescence, the strip of material is dissolved much sooner. This allows the scent and/or deodorizer to be dispersed throughout the toilet water much faster. In such a scenario, the strip may have a resulting texture, much like grit of sand paper.

In one embodiment a dissolvable effervescent toilet deodorizer comprises a water-soluble strip of material comprising a deodorizer. In one embodiment, the deodorizer may comprise ozone. In one embodiment, the deodorizer comprises a biocide. In one embodiment, the deodorizer comprises an essential oil.

In one method of use, a user would drop a dissolvable effervescent toilet deodorizer into the toilet bowl just prior to using the toilet. Once dropped into the toilet bowl, the dissolvable effervescent deodorizer begins to dissolve and effervesce. As a result of the effervescence, the dissolution occurs more rapidly and also disperses the deodorizer/scent into the toilet water and air to more effectively neutralize and/or prevent malodors.

In one method of use, a user would drop an effervescent tablet into a toilet before use, wherein the tablet dissolves and produces a pleasant fragrance, such as that emanating from an essential oil. The use of citric acid and sodium bicarbonate produces a fizzing (i.e., effervescence) in the toilet that aids in dissolution of the tablet, dispersing the deodorizer, and allows the user to know it's working. The use of citric acid and sodium bicarbonate is not required.

As appreciated from the foregoing, the dissolvable effervescent toilet deodorizer described herein solves the need for a toilet deodorizer that is easily transportable without leaking, may be easily used by a user prior to using or during use of a toilet, and that effectively eliminates malodors.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

What is claimed is:

1. A dissolvable effervescent toilet deodorizer, comprising:
a composition, the composition comprising:
citric acid;
sodium bicarbonate;
microcrystalline cellulose;
silica; and
an essential oil;
wherein the composition is pressed into tablet form.

2. A method of using the dissolvable effervescent toilet deodorizer of claim 1 to deodorize toilets, the method comprising dropping the dissolvable effervescent toilet deodorizer into the toilet immediately prior to using the toilet, the dissolvable effervescent toilet deodorizer dissolving and effervescing in the toilet bowl.

3. A method of using the dissolvable effervescent toilet deodorizer of claim 1 to deodorize toilets, the method comprising dropping the dissolvable effervescent toilet deodorizer into the toilet while using the toilet, the dissolvable effervescent toilet deodorizer dissolving and effervescing in the toilet bowl

4. A dissolvable effervescent toilet deodorizer, comprising:
a composition, the composition comprising:
citric acid;
sodium bicarbonate; and
an essential oil;
and, a water-soluble strip of material;
wherein the composition is affixed to, or embedded in, the water-soluble strip of material.

5. A method of using the dissolvable effervescent toilet deodorizer of claim 4 to deodorize toilets, the method comprising dropping the dissolvable effervescent toilet deodorizer into the toilet immediately prior to using the toilet, the dissolvable effervescent toilet deodorizer dissolving and effervescing in the toilet bowl.

6. A method of using the dissolvable effervescent toilet deodorizer of claim 1 to deodorize toilets, the method comprising dropping the dissolvable effervescent toilet deodorizer into the toilet while using the toilet, the dissolvable effervescent toilet deodorizer dissolving and effervescing in the toilet bowl.
